(19) United States
(12) Patent Application Publication
(10) Pub. No.: US 2025/0186529 A1
(43) Pub. Date: Jun. 12, 2025

(54) PHARMACEUTICAL COMPOSITION FOR MANAGING DYSMENORRHEA

(71) Applicant: HEMPSTREET MEDICARE PRIVATE LIMITED, Gurgaon (IN)

(72) Inventor: Pranay WAL, Kanpur (IN)

(21) Appl. No.: 18/845,000

(22) PCT Filed: Mar. 8, 2023
(86) PCT No.: PCT/IN2023/050221

§ 371 Date: Sep. 9, 2024

(30) Foreign Application Priority Data
Mar. 8, 2022 (IN) 202211012631

(51) Int. Cl. A61K 36/185; A61K 9/20; A61K 36/81; A61K 36/899

(52) U.S. Cl. CPC A61K 36/3482; A61K 9/2009; A61K 9/2013; A61K 9/2027; A61K 9/2059; A61K 36/81; A61K 36/899

(57) ABSTRACT

A pharmaceutical composition comprises Vijaya ranges from 55-65% of the weight of the composition, Vanshlochan ranges from 45-55% of the weight of the composition, Ashwagandha in the range of 10-20% by w/w and one or more of a pharmaceutically accepted excipients. The pharmaceutical composition is provided for managing dysmenorrhea.

### TECHNICAL FIELD

The present disclosure relates to a pharmaceutical composition provided for managing chronic menstrual pain. More particularly, the present disclosure relates to a pharmaceutical composition provided for managing dysmenorrhea.

### BACKGROUND

Dysmenorrhea is a medical term used to describe painful menstrual cramps or uterine contractions caused during menstruation. This can be further simplified as menstrual cramps or period cramps which take place in lower abdomen or pelvis and occur during or beginning of menstruation cycle. Conventional occurrences of Dysmenorrhea are present in 90% of the female population, wherein mild pain is experienced in initial 2 days of menstrual cycle while others experience severe abdominal pain along with nausea, dizziness, bloating, headache, diarrhea.

Dysmenorrhea is further classified into two categories: primary and secondary dysmenorrhea. In primary dysmenorrhea, cramps or pain are experienced before or during menstruation. The pain or cramps are caused by prostaglandins secreted by uterus lining. While secondary dysmenorrhea is caused by reproductive organ disorders. Secondary dysmenorrhea is characterized by the severe pain which may settle in before the beginning of menstruation, worsen during menstruation and may remain post menstruation.

Now-a-days, menstrual pain is a common problem, however the need for medication and the inability to function normally occurs less frequently. Nevertheless, at least one in four women experience distressing menstrual pain characterized by a need for medication and absenteeism from study or social activities. Menstrual pain was reported by 84.1% of women, with 43.1% reporting that pain occurred during every period, and 41% reporting that pain occurred during some periods. Periods are usually associated with mild, tolerable lower abdomen or back discomfort but the pain (dysmenorrhea) can be severe enough to interfere with day-to-day activities. The existing solutions in modern healthcare and allopathic medicine system are known to have harmful side effects in long term.

Hence, there is a need for development of pharmaceutical composition for managing dysmenorrhea.

### SUMMARY OF THE INVENTION

In an aspect of the present disclosure, a pharmaceutical composition includes Cannabis sativa in a range of 55-65% of the w/w, Vanshlochan in a range of 45-55% w/w, ashwagandha in a range of 10-20% by w/w, and one or more of pharmaceutically accepted excipients.

In another aspect of the present disclosure, the pharmaceutical composition includes one or more of pharmaceutically accepted excipient is selected from a group comprising a lubricant, a coating agent, a flowing agent, a binder, and an emulsifier.

In another aspect of the present disclosure, the lubricant is selected from a group including a magnesium stearate, talc, silica, vegetarian stearin, and stearic acid, calcium stearate, sodium lauryl sulphate, sodium benzoate, or PEG.

In another aspect of the present disclosure, the coating agent is selected from a group including aerosil powder, Shellac, cellulose acetate phthalate (CAP), polyvinyl acetate phthalate (PVAP), hyroxylpropyl cellulose, hyroxypropyl methylcellulose (HPMC) or Zein.

In another aspect of the present disclosure, the flowing agent is selected from a group including dibasic calcium phosphate, magnesium stearate, Colloidal Silicon dioxide (Aerosil), Cornstarch or Talc.

In another aspect of the present disclosure, the binder is selected from a group including acacia, starch, alginate, alginic acid, sucrose, gelatin, pregelatinized starch, PEG, sorbitol, and HPMC or PVP.

In another aspect of the present disclosure, the emulsifier is selected from a group including acacia, carbomer, Carboxymethylcellulose Sodium, Carmellose Sodium, Sodium Lauryl Sulfate or Carrageenan.

In another aspect of the present disclosure, the pharmaceutical composition is in a form selected from a group comprising a tablet, a capsule, a solution, a gel, an ointment, or a patch.

In another aspect of the present disclosure, the route of administration is selected from a group comprising an oral route, a sublingual route, a buccal route, an intravenous route, a subcutaneous route, an inhalation route, a nasal route, a cutaneous rote, a transdermal route.

Another aspect of the present disclosure, a pharmaceutical composition includes 62.5 mg Cannabis sativa by w/w, 10.5 mg Withania somnifera by w/w, and 52 mg Bambusa arundinacea of by w/w and one or more of pharmaceutically accepted excipient.

Another aspect of the present disclosure, a pharmaceutical composition includes 60 gm of Cannabis sativa by w/w, 15.5 mg Withania somnifera by w/w, and 49.5 mg of Bambusa arundinacea by w/w and one or more of pharmaceutically accepted excipient.

### BRIEF DESCRIPTION OF DRAWINGS

The drawing/s mentioned herein disclose exemplary embodiments of the claimed invention. Other objects, features, and advantages of the present invention will be apparent from the following description when read with reference to the accompanying drawing:

FIG. 1 is a graphical representation that illustrates the λmax value of the herbal extract.

FIG. 2 is a graphical representation that illustrates the FTIR spectra of pure herbal extract and one or more of pharmaceutically accepted excipients.

FIG. 3 is a graphical representation that illustrates the effect of different dose of the aqueous extract of TVV on serum creatinine concentration.

FIG. 4 is a graphical representation that illustrates the effect of different doses of the aqueous extract of TVV on body weight.

FIG. 5 is a graphical representation that illustrates the effect of different does of aqueous extract of TVV on serum liver enzyme activity.

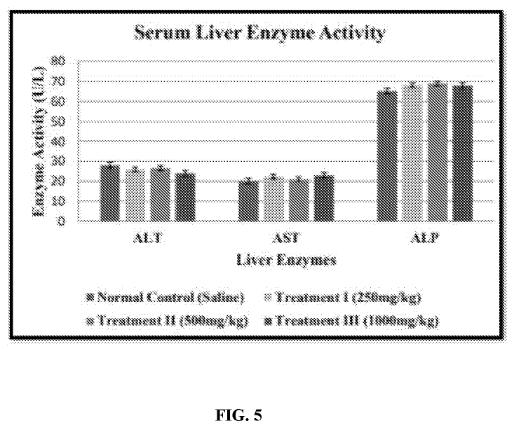

### DETAILED DESCRIPTION OF PREFERRED EMBODIMENTS

This section is intended to provide explanation and description of various possible embodiments of the present invention. The embodiments used herein, and the various features and advantageous details thereof are explained more fully with reference to non-limiting embodiments illustrated in the accompanying drawing/s and detailed in the following description. The examples used herein are intended only to facilitate understanding of ways in which the embodiments may be practiced and to enable the person skilled in the art to practice the embodiments used herein. Also, the examples/embodiments described herein should not be construed as limiting the scope of the embodiments herein.

As mentioned, there is a need for development of compositions for managing dysmenorrhea. The embodiments herein provide Cannabis sativa based pharmaceutical composition for managing dysmenorrhea.

### Definitions

Vijaya (Cannabis sativa) is an annual herbaceous plant, indigenous to India and the South-East Asia. The Cannabis sativa of the present disclosure is sourced from Rama shiv pvt ltd, Unnao, Uttar Pradesh, India. The Cannabis sativa is used for managing pain, asthma, insomnia, depression, spasm, and loss of appetite.

Ashwagandha (Withania somnifera) is an herb, sourced from Block-Z, 26/27 Commercial Enclave, Mohan Garden, Uttam Nagar, Delhi, India. The herb is used for relieving stress, improving energy levels and countering anxiety. Its mood-boosting potential have also been validated through clinical studies.

Vanshlochan (Bambusa arundinacea) also known as Tabasheer, is siliceous resin and bamboo exudate, which is a form of herbal silica obtained from bamboo trees. The herb is sourced from Rama shiv pvt ltd, Unnao, Uttar Pradesh India. Vanshlochan has healing properties, and is particularly useful in providing strength, flexibility and resilience to collagen and elastin connective tissues. Vanshlochan is also used as stimulant, diuretic, coolant, sweet tonic, aphrodisiac, and expectorant.

As used herein, “pharmaceutically accepted excipient” refers to the compounds other than the active pharmaceutical ingredients that are pharmaceutically evaluated as fit for utilization. They may include binders, emulsifiers, additives, stabilizing agents, lubricants, disintegrating agents, gliding agents, and others that are known to a person skilled in art.

As used herein, “herbal extract” refers to the extracts of Cannabis sativa, Bambusa arundinacea and Withania somnifera.

The term “Vijaya” and “Cannabis sativa” are interchangeably used across the disclosure.

The term “Vanshlochan”, “bamboo” and “Bambusa arundinacea” are interchangeably used across the disclosure.

The term “Ashwagandha” and “Withania somnifera” are interchangeably used across the disclosure.

Cannabis sativa is known to activate cannabinoid receptors of the brain thereby reducing pain levels. Withania somnifera is known analgesic that blocks the pain signals to travel towards nervous system. The anti-inflammatory properties of Bambusa arundinacea help in menstrual pain.

The present pharmaceutical composition includes Cannabis sativa, Bambusa arundinacea and Withania somnifera that produces a synergistic effect. The Bamboo silicate activates the anti-inflammatory effect of cannabis. Cannabis sativa allows for better absorption of the medication when orally ingested and ashwagandha being a mood enhancer, helps with the “feeling” part of the ailment.

The pharmaceutical combination is a high absorption, anti-inflammation, pain relief and mood enhancement formulation. This helps improve general quality of life and not just suppress individual symptoms.

The present pharmaceutical composition described herein includes active ingredient(s) for dysmenorrhea that alleviate pain with no observed side-effects to women. The present pharmaceutical composition includes Cannabis sativa ranging between (55-65%) of the weight of the composition, Bambusa arundinacea ranging between (45-55%) of the weight of the composition, Withania somnifera ranging between (10-20%) by weight of the composition and one or more pharmaceutically acceptable excipients.

In an embodiment, Cannabis sativa is used in powdered form. In another embodiment, the extract of Cannabis sativa may be obtained from a process which is selected from a group, but not limited to, a Soxhlet maceration, dynamic maceration, ultrasonic-assisted extraction, microwave-assisted extraction, supercritical fluid extraction, and a pressurized liquid extraction. In another embodiment, the Cannabis sativa extract is freeze dried to obtain powder.

In an embodiment, Bambusa arundinacea may be used in powdered form. In another embodiment, Bambusa arundinacea juice may be used in the pharmaceutical composition. In another embodiment, Bambusa arundinacea shoot may be grinded to form a powder.

In an embodiment, Withania somnifera extract is used in the pharmaceutical composition. In another embodiment, the extract is selected from a group, but not limited to, a powdered extract, aqueous extract, and oil-based extract.

In a preferred embodiment, the pharmaceutical composition of the present invention is in the form of a tablet. In another embodiment, the pharmaceutical composition is available in the form of a gel. In another embodiment, the pharmaceutical composition is available in the form of a water-soluble pill. In another embodiment, the pharmaceutical composition is in a powdered form. In another embodiment, the pharmaceutical composition is available in form of an ointment. In another embodiment, the pharmaceutical composition is in form of a polyherbal tablet. In another embodiment, the pharmaceutical composition is available as a liquid extract. In another embodiment, the pharmaceutical composition is available as water soluble hard gelatin capsule. In another embodiment, the pharmaceutical composition is available as water soluble soft gelatin capsule. In another embodiment, the pharmaceutical composition is available as a patch.

In an embodiment, the route of administration of the pharmaceutical composition is selected from a group, but not limited to, an oral route, a sublingual route, a buccal route, an intravenous route, a subcutaneous route, an inhalation route, a nasal route, a cutaneous rote, a transdermal route.

In an embodiment, the pharmaceutically accepted excipient is selected from a group, but not limited to, a binder, an additive, a thickening agent, a gliding agent, an emulsifier, a disintegrating agent, a lubricant, and a coating agent.

In another embodiment, the thickening agent is selected from a group, but not limited to, modified starch, corn starch, pre-gelatinized starch, agar-agar, sodium alginate, gelatin, acacia gum, gum tragacanth, pectin, carrageenan, xanthan gum, epicatechin or alginic acid.

In another embodiment, the disintegrating agent is selected from a group, but not limited to, starch, microcrystalline cellulose, sodium alginate, crospovidone, croscarmellose Sodium, sodium starch glycolate, calcium alginate, powdered cellulose, chitosan hydrochloride, corn starch, pregelatinized starch, docusate sodium, magnesium aluminum silicate, and methylcellulose.

In another embodiment, the lubricant is selected from a group, but not limited to, talc, silica, vegetable stearin, stearic acid, magnesium stearate, calcium stearate, sodium lauryl sulphate, sodium benzoate, and PEG.

In another embodiment, the coating agent is selected from a group, but not limited to, aerosil powder, Shellac, cellulose acetate phthalate (CAP), polyvinyl acetate phthalate (PVAP), hyroxylpropyl cellulose, hyroxypropyl methylcellulose (HPMC), and Zein.

In another embodiment, the flowing agent is selected from a group, but not limited to, a dibasic calcium phosphate, magnesium stearate, Colloidal Silicon dioxide (Aerosil), Cornstarch, and Talc.

In another embodiment, the binder is selected from a group comprising acacia, starch, alginate, alginic acid, sucrose, gelatin, pregelatinized starch, PEG, sorbitol, and HPMC, and PVP.

In another embodiment, the emulsifier is selected from a group comprising acacia, carbomer, Carboxymethylcellulose Sodium, Carmellose Sodium, Sodium Lauryl Sulfate, and Carrageenan.

In an embodiment, the pharmaceutical composition is further blended with natural herbs for reducing pain. In another embodiment, essential oils are added to the pharmaceutical composition for boosting mood.

In an embodiment, the pharmaceutical composition is provided for managing dysmenorrhea. In another embodiment, the pharmaceutical composition is provided for managing primary dysmenorrhea. In another embodiment, the pharmaceutical composition is provided for managing secondary dysmenorrhea.

In an embodiment, the pharmaceutical composition includes 62.5 mg Cannabis sativa by w/w, 10.5 mg Withania somnifera by w/w, and 52 mg of Bambusa arundinacea by w/w and one or more of pharmaceutically accepted excipient.

In another embodiment, the pharmaceutical composition includes 60 mg of Cannabis sativa by w/w, 15.5 mg Withania somnifera by w/w, and 49.5 mg of Bambusa arundinacea by w/w and one or more of pharmaceutically accepted excipient.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices, and materials are described herein. It is to be understood that this disclosure is not limited to particular methods, and experimental conditions described, as such methods and conditions may vary.

### Example 1: Physiochemical Standardization of Herbal Extract

250 gram (g) of the Cannabis sativa was weighed, spread in a thin layer, and inspected with an unaided eye or magnifying glass. The sample was then separated manually and weighed. The percentage of foreign organic matter was weighed and determined with reference to the weight of the drug taken.

The above steps were also repeated for Bambusa arundinacea and Withania somnifera.

### Example 2: Determination of Moisture Content of Herbal Extract

5 g of air-dried material was prepared and accurately weighed in a previously dried and tarred petri dish. The samples of Cannabis sativa, Bambusa arundinacea and Withania somnifera were distributed evenly and placed in the drying oven and heated to 105° Celsius (C), the petri dishes were further removed from the oven and then kept in the desiccator. The petri dishes were allowed to cool and then weighed.

### Example 3: Determination of Total Ash Value of Herbal Extract

The residue remaining after incineration infers the ash content of the drug, which simply represents inorganic salts, naturally occurring in drug, adhering to it, or deliberately added to it as a form of adulteration. Ash value is utilized as a criterion to judge the identity or purity of crude drugs. Total ash usually consists of carbonates, phosphates, silicates, and silica.

2 g of the Cannabis sativa was weighed and spread as a fine layer at the bottom of the crucible. Further, the crucible was incinerated at a maximum temperature of 450° C. until free from carbon. The crucible was allowed to cool and then weighed. The above steps were also repeated for Bambusa arundinacea and Withania somnifera.

### Example 4: Determination of Total Acid Insoluble Ash Value of Herbal Extract

Post incineration of Cannabis sativa the ash produced was boiled with 10%, 25 milliliter (ml) of hydrochloric acid for 5 minutes (mins). The insoluble ash accumulated on an ash less filter paper by filtration and washed with hot water and further transferred into a tarred silica crucible. At first it was ignited ten allowed to cool and further weighed. The procedure was repeated till a constant weight was observed. The percentage of acid insoluble ash was calculated with reference to the air-dried drug.

The above steps were also repeated for Bambusa arundinacea and Withania somnifera.

### Example 5: Determination of Water-Soluble Ash of Herbal Extract

25 ml of water was added to the ash produced post incineration of Cannabis sativa and boiled for 5 minutes. The insoluble matter was collected in a sintered glass crucible or an ashless filter paper. Further, it was washed with hot water and ignited in the crucible for 15 minutes at a maximum temperature of 450° C.

The above steps were also repeated for Bambusa arundinacea and Withania somnifera.

### Example 6: Determination of Extractive Values of Herbal Extract

Extractive value enacts as a measure of the content of the drug extracted by solvents. Extractive value may be aqueous soluble, alcohol soluble and other non-aqueous solvent soluble. Implementation of such method determines the number of active constituents in a given amount of medicinal plant material when extracted with solvents. The extraction of any drug with solvents yields a solution containing different phytoconstituents.

6.1 Aqueous Extract of Cannabis sativa, Bambusa Arundinacea and Withania somnifera

### Methodology

2 gm of previously weighed air-dried powdered of Cannabis sativa was taken in a glass stoppered flask and macerated with 100 ml of chloroform water (1:99). It was shaken frequently for 6 hours (hrs.) and then allowed to stand for 18 hrs. Further it was filtered rapidly taking precautions against loss of the solvent. Further, 10 ml of filtrate was evaporated to dryness in a tarred flat-bottomed Petri dish in triplicates, dried at 105° C., cooled in a desiccator and weighed. The percentage of aqueous-soluble extractive was calculated with reference to air-dried drug. The above steps were also repeated for Bambusa arundinacea and Withania somnifera.

6.2 Alcohol soluble extract of Cannabis sativa, Bambusa arundinacea and Withania somnifera

### Methodology:

2 gm of previously weighed air-dried powder of Cannabis sativa, was taken in a glass stoppered flask and macerated with 100 ml of ethanol. It was shaken frequently for 6 hrs. and then allowed to stand for 18 hrs. It was filtered rapidly taking precautions against loss of the solvent. 10 ml of filtrate was evaporated to dryness in a tarred flat-bottomed Petri dish in triplicates, dried at 105° C., cooled in desiccators and weighed. The percentage of ethanol-soluble extractive was calculated with reference to air-dried drug. The above steps were also repeated for Bambusa arundinacea and Withania somnifera.

TABLE 1
Result:

| | Cannabis | Withania | Bambusa |
|---|---|---|---|
| Test | sativa | somnifera | arundinacea |
| Foreign matter | 0% | 0.1% | 0.4% |
| Loss on drying | 1% | 3.6% | 2.9% |
| Total ash value | 14.78% | 5.78% | 6.88% |
| Total acid | 15.25% | 1.87% | 4.1% |
| insoluble ash | | | |
| Total aqueous | 24.35% | 1.15% | 5.85% |
| soluble ash | | | |
| Aqueous soluble | 22.5% | 9.5% | 30% |
| extractive | | | |
| Alcohol soluble | 5% | 6% | 18.5% |
| extractive | | | |

### 2. Preformulation Studies

### 2.1 Physical Identification of Herbal Extract

Cannabis sativa-Cannabis sativa in the form of powder was procured from Rama shiv pvt ltd. It was brown in color.

Withania somnifera—The extract of Withania somnifera in the form of powder was procured from Block-Z, 26/27 Commercial Enclave, Mohan Garden, Uttam Nagar, Delhi, 110059. It was brown fine powder with characteristic odor. (Vital herbs)

Bambusa arundinacea—The extract of Bambusa arundinacea in the form of powder was procured from Rama shiv pvt ltd, Unnao, Uttar Pradesh, India and it was white in color and crystalline form.

### 2.2 Melting Point

The melting points of Withania somnifera, Cannabis sativa and Bambusa arundinacea are discussed in Table 2 below.

TABLE 2

| Herbal extract | Melting point | |
|---|---|---|
| Withania somnifera | 186° | C. |
| Cannabis sativa | 70° | C. |
| Bambusa arundinacea | 180° | C. |

### 2.3 Compositions of Polyherbal Tablet

Two compositions were prepared for the evaluation of different parameters. The two compositions are named as F1 and F2 respectively. The general composition 5 of the pharmaceutical composition is described in Table 3 below. The compositions for F1 and F2 are described in Table 4 and 5, respectively.

TABLE 3
Composition for general formulation

| Ingredients | In % | In mg |
|---|---|---|
| Herbs | 18.2 | 125 |
| Magnesium stearate | 0.29 | 1.992 |
| Aerosil powder | 0.14 | 0.962 |
| DBDC | 33.63 | 230.974 |
| Sodium Benzoate | 0.25 | 1.717 |
| Starch slurry | 45.27 | 310.919 |
| Gum acacia slurry | 1.11 | 7.624 |
| PVP slurry | 1.11 | 7.624 |
| Total | 100 | 686.811 |

TABLE 4
Composition for F1 formulation

| S no. | Ingredients | Quantity in mg |
|---|---|---|
| 1 | Cannabis sativa | 62.5 |
| 2 | Withania | 10.5 |
| | somnifera | |
| 3 | Bambusa | 52 |
| | arundinacea | |
| 4 | Magnesium | 1.992 |
| | stearate | |
| 5 | Aerosil powder | 0.962 |
| 6 | DBDC | 230.974 |
| 7 | Sodium | 1.717 |
| | Benzoate | |
| 8 | Starch slurry | 310.919 |
| 9 | Gum acacia | 7.624 |
| | slurry | |
| 10 | PVP slurry | 7.624 |
| 11 | Total | 686.811 |

TABLE 5
Composition for F2 formulation

| | | Quantity |
|---|---|---|
| S no. | Ingredients | in mg |
| 1 | Cannabis sativa | 60 |
| 2 | Withania | 15.5 |
| | somnifera | |
| 3 | Bambusa | 49.5 |
| | arundinacea | |
| 4 | Magnesium | 1.992 |
| | stearate | |
| 5 | Aerosil powder | 0.962 |
| 6 | DBDC | 230.974 |
| 7 | Sodium Benzoate | 1.717 |
| 8 | Starch slurry | 310.919 |
| 9 | Gum acacia slurry | 7.624 |
| 10 | PVP slurry | 7.624 |
| 11 | Total | 686.811 |

### 2.3 UV Spectroscopy

### 2.3.1 Determination of λMax

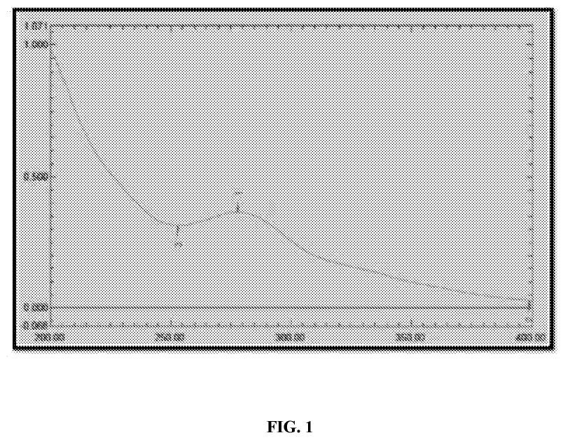

100 milligram (mg) of Cannabis sativa, Bambusa arundinacea and Withania somnifera was properly weighed and diluted in 100 ml phosphate buffer pH 2.0, yielding a 1 mg/ml solution. It was diluted to a concentration of 500 micro-gram (μg)/ml and used as a stock solution. The spectra were then captured across a wavelength range of 200-400 nanometer (nm). The results of the study are illustrated in FIG. 1

### 2.3.2 Preparation of Phosphate Buffer pH6.8

0.136 gm of potassium dihydrogen phosphate was dissolved in 8000 ml of distilled water and place in a volumetric flask of 1000 ml then the pH was adjusted to 2.0 with hydrochloric acid. Further, 1000 ml of water was added to make phosphate buffer. (I.P. volume. 1, 2014 2.)

### 2.3.3 Preparation of Calibration Curve of Herbal Extract

Standard stock solution was prepared by dissolving 100 mg of powder of Cannabis sativa, Bambusa arundinacea and Withania somnifera, respectively in 100 ml of 6.8 pH phosphate buffer to make final concentration. Individual 1 ml was taken from the stock mixture and diluted with phosphate buffer to makeup volume 10 ml and prepares series of concentration ranging from 500 to 900 μg/ml. The absorption maxima were measured between 200 to 400 nm. The data for the calibration curve of F1 and F2 in phosphate buffer is shown in Table 6 and 7 respectively.

TABLE 6
Data for the calibration curve of F1 in phosphate buffer pH 2.0

| | Concentration | Absorbance at |
|---|---|---|
| S. no | in μg/ml | 254 λₘₐₓ |
| 1 | 500 | 0.334 |
| 2 | 600 | 0.398 |
| 3 | 700 | 0.439 |
| 4 | 800 | 0.496 |

TABLE 7
Data for the calibration curve of F2 in phosphate buffer pH 2.0

| | Concentration | Absorbance at |
|---|---|---|
| S. no | in μg/ml | 254 λₘₐₓ |
| 1 | 500 | 0.321 |
| 2 | 600 | 0.347 |
| 3 | 700 | 0.375 |
| 4 | 800 | 0.401 |
| 5 | 900 | 0.425 |

### Example 3 Herbal Extract-Excipients Compatibility Studies

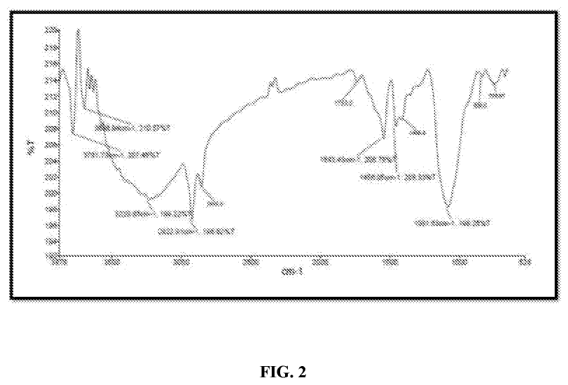

Herbs-excipients compatibility study identify parameters for the development of formulation in its dosage form to increase bioavailability and proper administration of the drug. Compatibility between the drug and excipients determine the effectiveness of delivery system. The degree of interaction between herbs and excipients may affect the performance related characteristic of the formulation. The type, nature, and concentration of excipients are dependent on the desired formulation which results in a lack of vehicle for utilization in delivery as a universal excipient for all herbs. The result of the study is illustrated in FIG. 2.

### Example 4 Pre-Compression Study

### 4.1 Granulation

Granules are prepared through the wet granulation technique. First, the preparation of the slurry is conducted by adding Gum acacia, PVP, and Starch in a weighed amount sufficient water to form slurry-like consistency. The ingredients are properly mixed, and slurry is added with continuous mixing to form the granules. The granules are then dried.

The formulations F1 and F2 were tested for different parameters such as bulk density, tapped density, angle of response, Hausner ratio, Interday pH and Intraday pH. The results of the above parameters are described in Table 8.

TABLE 8
Parameters of different formulation

| | Bulk | Tapped | | | | |
|---|---|---|---|---|---|---|
| | density | density | Angle of | Hausner | Interday | Intraday |
| Formulation | (gm/ml) | (gm/ml) | repose (Θ) | ratio | pH | pH |
| F1 | 0.35 ± 0.02 | 0.4 ± 0.03 | 24.34° ± 0.1 | 1.142 ± 0.02 | 7.41 ± 0.04 | 7.43 ± 0.04 |
| F2 | 0.392 ± 0.01 | 0.476 ± 0.16 | 23.12° ± 0.26 | 1.214 ± 0.03 | 7.40 ± 0.03 | 7.39 ± 0.03 |

### Example 4.2 Weight Variation

To calculate the average weight of the tablet, 20 tablets were prepared, and the average weight of the tablet were calculated based on the following formula:

Standard formula for calculating the average weight of tablet:

Total weight of the tablet/total no. of tablets

The weight variation for formulation F1 and F2 are described in Table 9 and 10 respectively.

TABLE 9
Weight variation of F1 formulation

| | Weight (mg) | | |
|---|---|---|---|
| | 1st | 2nd | 3rd |
| Tablet | time | time | time |
| T1 | 687 | 688 | 687 |
| T2 | 684 | 684 | 685 |
| T3 | 683 | 685 | 682 |
| T4 | 687 | 686 | 690 |
| T5 | 690 | 681 | 688 |
| T6 | 682 | 682 | 684 |
| T7 | 687 | 686 | 687 |
| T8 | 681 | 682 | 681 |
| T9 | 684 | 685 | 685 |
| T10 | 687 | 688 | 687 |
| T11 | 685 | 685 | 686 |
| T12 | 685 | 683 | 687 |
| T13 | 691 | 689 | 689 |
| T14 | 689 | 687 | 687 |
| T15 | 682 | 683 | 683 |
| T16 | 687 | 687 | 686 |
| T17 | 684 | 684 | 685 |
| T18 | 681 | 682 | 681 |
| T19 | 687 | 688 | 688 |
| T20 | 690 | 690 | 689 |
| Total | 13713 | 13705 | 13717 |

The weight of tablet for F1 formulation was observed more or equal 5 to 250 mg equates to weight variation limit of ±5%. The weight variation allows 685.58±34.58 for F1 formulation.

TABLE 10
Weight variation of F2 formulation

| | weight (mg) | | |
|---|---|---|---|
| | 1st | 2nd | 3rd |
| Tablet | time | time | time |
| T1 | 690 | 692 | 691 |
| T2 | 684 | 684 | 685 |
| T3 | 686 | 687 | 686 |
| T4 | 685 | 684 | 685 |
| T5 | 688 | 688 | 687 |
| T6 | 685 | 686 | 684 |
| T7 | 681 | 682 | 681 |
| T8 | 687 | 687 | 688 |
| T9 | 686 | 688 | 687 |
| T10 | 684 | 683 | 683 |
| T11 | 689 | 689 | 688 |
| T12 | 692 | 691 | 691 |
| T13 | 690 | 689 | 690 |
| T14 | 685 | 685 | 686 |
| T15 | 687 | 687 | 689 |
| T16 | 690 | 692 | 691 |
| T17 | 691 | 691 | 692 |
| T18 | 681 | 683 | 682 |
| T19 | 687 | 686 | 687 |
| T20 | 688 | 687 | 687 |
| Total | 13736 | 13741 | 13740 |

The weight variation allows 686.95±34.34 for F2 formulation.

The tablets of both the formulation F1 and F2 were tested for other parameters such as friability of the tablet, disintegration time of the tablet, hardness of the tablet, thickness of the tablet and diameter of the tablet. The results of the parameters are discussed in Table 11.

TABLE 11
Other parameters

| | Friability | Disintegration | Hardness | Thickness | Diameter |
|---|---|---|---|---|---|
| Formulation | (%) | time (min) | (kg/cm²) | (mm) | (mm) |
| F1 | 0.097 ± 0.01 | 1 min 36 sec | 5.367 ± 0.58 | 63.66 | 101 |
| F2 | 0.099 ± 0.03 | 3 min 45 sec | 5.667 ± 0.725 | 63.66 | 101 |

### Example 4.4 In Vitro Drug Release

The test results for in vitro drug release of formulation F1 and F2 are discussed in Table 12 and 13 respectively.

TABLE 12
In-vitro drug release of F1 formulation

| | | Conc. in | % Drug | Log % | Sq. rt. | Cube root | Log |
|---|---|---|---|---|---|---|---|
| Time | Absorbance | DF (mg) | Rel. (%) | DR | of time | of % DR | Time |
| 1.00 | 0.08 | 21.24 | 16.99 | 1.23 | 1.00 | 2.57 | 0.00 |
| 2.00 | 0.12 | 78.84 | 63.07 | 1.80 | 1.41 | 3.98 | 0.30 |
| 3.00 | 0.14 | 120.24 | 96.19 | 1.98 | 1.73 | 4.58 | 0.48 |

TABLE 13
In-vitro drug release of F2 formulation

| | | Conc. in | % Drug | Log % | Sq. rt. | Cube root | Log |
|---|---|---|---|---|---|---|---|
| Time | Absorbance | DF (mg) | Rel. (mg) | DR | of time | of % DR | Time |
| 1.00 | 0.19 | 39.60 | 31.68 | 1.50 | 1.00 | 3.16 | 0.00 |
| 2.00 | 0.21 | 72.60 | 58.08 | 1.76 | 1.41 | 3.87 | 0.30 |
| 3.00 | 0.21 | 99.60 | 79.68 | 1.90 | 1.73 | 4.30 | 0.48 |
| 4.00 | 0.22 | 117.60 | 94.08 | 1.97 | 2.00 | 4.55 | 0.60 |

TABLE 14
Release kinetic parameter

r2

| S. no | Model name | F1 | F2 |
|---|---|---|---|
| 1 | Zero order | 0.9912 | 0.9836 |
| 2 | First order | 0.9193 | 0.9285 |
| 3 | Higuchi | 0.9997 | 0.9985 |
| 4 | Korsmeyer peppas | 0.9811 | 0.9948 |

### Example 5: Animal Model Study

Young healthy wistar mice (female), 5-6 weeks old, weighing about 24-25 g and Wistar rats (both sexes), 9-12 weeks old weighing about 150-200 g was used in this study. The animals were purchased from breeder house LITR, Lucknow. The animals were maintained under standard environmental conditions (23-25° C., 12 h/12 h light/dark cycle) and had free access to standard pelleted diet, water ad libitum. Animals were acclimatized to laboratory environment for a week prior to start study.

### Example 5.1: Dose Calculation

Toxicity studies were performed for dose calculation. The dose used for conducting the toxicity studies were chosen on the basis of polyherbal tablet formulation ((2 g/day for adults). Higher doses (1000 and 2000 mg/kg/day) were selected for sub-acute toxicity study while the dose of the individual rats in all different groups were calculated based on the body weights before the start of the study.

### Example 5.2: Acute Toxicity Study

Acute oral toxicity study was performed as per the guidelines set by the Organization for Economic Co-operation and Development (OECD)-423: Acute Oral Toxicity-Acute Toxic Class Method. The safety aspect of the aqueous extract of TVV was evaluated and found to be non-toxic for dose level up to 2000 mg/kg body weight. Signs of toxicity and mortality were observed for the first 30 minutes followed by 1, 2, 4, 6 and 24 hours and thereafter twice daily until 2nd-14th day. The wellness parameters such as discoloration, fur loss, nasal and oral mucous membrane for any ulceration, respiratory rate, heart rate, salivation, lacrimation, lethargy, piloerection, urinary incontinence, defecation, sleep, gait, tremors, convulsion, and mortality were all observed, recorded for each animal and compared between control and experimental groups. The observations are tabulated in table 5.3. No death was observed in dose above 2000 mg/kg. Thus, according to the Globally Harmonized System (GHS) for the classification of chemicals which cause acute toxicity, the ethanolic extract of PHP was classified under Category 5. Mice were fasted for 24 h prior to the commencement of this test. Ten animals (mice); five males and five females were used, and each animal were given a single dose of 2000 mg/kg of Polyherbal product (p.o.). Animals were observed strictly and individually for first 30 min after dosing and periodically during first 24 h. (with special attention during first 4 h) and daily thereafter for 3 days. Mice were observed for altered autonomic effects (lacrimation, salivation, piloerection), central nervous system effect (tremors, convulsion, drowsiness) skin (fur), body weight, food consumption, water consumption and mortality.

### Results

There were no toxic signs observed in clinical parameters during acute study, which indicates that the LD50 of the polyherbal formulation is greater than 2000 mg/kg/day.

### Example 5.2 Sub-Acute Toxicity Study

From the observation of acute toxicity studies of aqueous extract of TVV, the dose for repeated dose toxicity study was selected as 250 mg/kg, 500 mg/kg and 1000 mg/kg. The Aqueous extract of TVV was orally administered to the Wistar rats in graduated doses to the experimental groups, one dose level per group for a period of 28 days. On the 29th day, the blood of Wistar rats was collected from the experimental groups. Serum was separated by centrifugation for about 3000 rpm. The serum was collected using micropipette from the samples and analyzed according to the standard procedures from the kit available in the college. Forty Wistar rats were divided into 4 groups of 10 each (5 males & 5 females). Three groups of experimental doses 250 mg/kg/day, 500 mg/kg/day and 1000 mg/kg/day respectively and the fourth group was of control. Control group was fed with only normal food and water. Animals were weighed weekly and observed for behavioral changes, food and water consumption, and general morphological changes. On the 28th day of study period, animals were anaesthetized by i.p. administration of 5 ml/kg of a solution of 1% chloralose in 25% urethane (w/v). Blood samples were collected from rats by cardiac puncture into EDTA sample tubes for hematological analysis and into heparinized tubes for serum generation for biochemical analysis. Serum was acquired after allowing blood to congeal for 30 min. And centrifugation. After sacrificing the experimental animals, vital organs including kidneys and Liver were harvested, carefully examined. The off cuts of the organs were conserved for histopathological assessment. Mortality in each study group was recorded during the 28-day administration of the product.

### 5.2.1 Hematological Analysis

Blood samples were analyzed by using established procedures and the CBC machine. Parameters evaluated include WBCs count, RBCs count, PLT count, Hb, HCT, MCV, MCH and MCHC.

### 5.2.2 Biochemical Parameters

Serum samples were analyzed for creatinine, blood urea, uric acid, TG, cholesterol, HDL, VLDL, bilirubin, AST and ALT by using instrument.

### 5.2.3 Histopathological Assessment

Liver and kidney tissues were obtained from experimental animals and fixed in 10% formol-saline. Later, these tissues were dehydrated in graded alcohol, inserted in paraffin, and cut into 4-5 μm thick sections. Hematoxylin-eosin was used to stain the sections for photo microscopic assessment using a Model N-400ME photomicroscope. Slides were examined using the 40×, and 100× objectives

### 5.2.4 Statistical Analysis

Results are communicated as mean±SEM. Data analysis was carried out using One-way ANOVA with post-hoc Tukey's HSD test. For weight variation, Two-way ANOVA with post-hoc Dunnett's test was applied for multiple comparisons (SPSS 21). Significance was measured at values of p<0.05 and p<0.01.

### Observation

The study was conducted for four weeks (28 days) with three different doses; 250 mg/kg/day, 500 mg/kg/day, 1000 mg/kg/day and one group was taken as control. The parameters focused were body weight, food and water consumption, hematological parameters (WBCs, RBCs, PLT, Hb, HCT, MCH, MCV, and MCHC), liver function parameters (Bilirubin, ALT, AST), renal function profile (blood urea, creatinine, and uric acid), and lipid profile (triglycerides, cholesterol, HDL, VLDL)

### Results

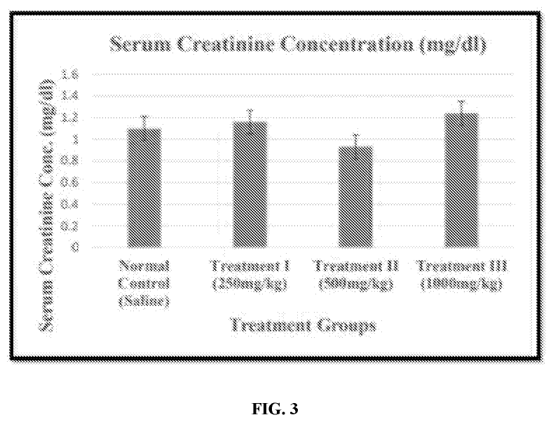

The findings of acute and sub-acute toxicitydy revealed that this polyherbal TVV Plus formulation is non-toxic with single oral dose of 1000 mg/kg/day. The 28 days sub-acute toxicity study revealed no significant changes with 250 mg/kg/day. Slight changes in biochemical parameters and structural levels were at 500 mg/kg/day and mild to severe cellular changes at 1000 mg/kg/day. So, it is concluded that the formulation is safe to use at dose of 500 mg/kg/day for a period of 28 days. The effect of different dose of the aqueous extract of TVV on serum Creatinine concentration is shown in FIG. 3.

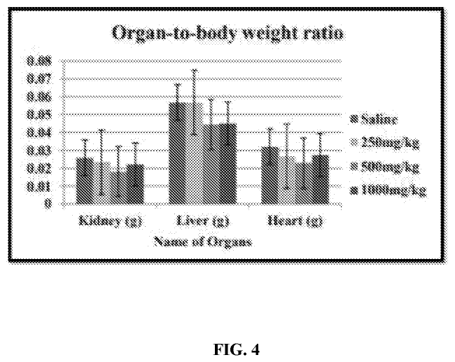

Weight variations of both male and female treated, and control groups were noted. A gradual raise in the weights of male rats throughout the sub-acute study. There was a significant increase in the weights observed from day 14 till the end of the study in all treatment groups in comparison to their relevant weights on day 1. Whereas, for females a different pattern was observed. The graphical representation shows that at the start of the study the body weights of the treated groups were normal, no significant change in the weight was observed till day 14 as compared to their relevant weights on day 1. On day 21 there was a significant increase in weight of control, 500 and 1000 mg/kg/day treatment groups but there was not any marked change for treatment group of 250 mg/kg/day. While on day 28, all groups i.e. control, 500 and 1000 mg/kg/day except 250 mg/kg/day showed significant changes in weights for female rats The effect of different does of aqueous extract of TVV on body weight of rats are shown in FIG. 4.

We claim:

1. A pharmaceutical composition, the pharmaceutical composition comprises:
a) Cannabis sativa in a range of 55-65% of the w/w;
b) Bambusa arundinacea in a range of 45-55% w/w;
c) Withania somnifera in a range of 10-20% by w/w; and
d) one or more of pharmaceutically accepted excipients.

2. The pharmaceutical composition as claimed in claim 1, wherein the one or more of pharmaceutically accepted excipient is selected from a group comprising a lubricant, a coating agent, a flowing agent, a binder, and an emulsifier.

3. The pharmaceutical composition as claimed in claim 3, wherein the lubricant is selected from a group comprising a magnesium stearate, talc, silica, vegetarian stearin, and stearic acid, calcium stearate, sodium lauryl sulphate, sodium benzoate, or PEG.

4. The pharmaceutical composition as claimed in claim 3, wherein the coating agent is selected from a group comprising aerosil powder, Shellac, cellulose acetate phthalate (CAP), polyvinyl acetate phthalate (PVAP), hyroxylpropyl cellulose, hyroxypropyl methylcellulose (HPMC) or Zein.

5. The pharmaceutical composition as claimed in claim 3, wherein the flowing agent is selected from a group comprising dibasic calcium phosphate, magnesium stearate, Colloidal Silicon dioxide (Aerosil), Cornstarch or Talc.

6. The pharmaceutical composition as claimed in 3, wherein the binder is selected from a group comprising acacia, starch, alginate, alginic acid, sucrose, gelatin, pregelatinized starch, PEG, sorbitol, and HPMC or PVP.

7. The pharmaceutical composition as claimed in claim 3, wherein the emulsifier is selected from a group comprising acacia, carbomer, Carboxymethylcellulose Sodium, Carmellose Sodium, Sodium Lauryl Sulfate or Carrageenan.

8. The pharmaceutical composition as claimed in claim 1, is in a form selected from a group comprising a tablet, a capsule, a solution, a gel, an ointment, or a patch.

9. The pharmaceutical composition as claimed in claim 1, route of administration is selected from a group comprising an oral route, a sublingual route, a buccal route, an intravenous route, a subcutaneous route, an inhalation route, a nasal route, a cutaneous rote, a transdermal route.

10. A pharmaceutical composition, the pharmaceutical composition comprises:
a) Cannabis sativa 62.5 mg by w/w;
b) Withania somnifera 10.5 mg by w/w;
c) Bambusa arundinacea 52 mg of by w/w; and
d) one or more of pharmaceutically accepted excipients.

11. A pharmaceutical composition, the pharmaceutical composition comprises:
a) Cannabis sativa 60 gm of by w/w;
b) Withania somnifera 5.5 mg by w/w;
c) Bambusa arundinacea 49.5 mg by w/w; and
d) one or more of pharmaceutically accepted excipients.
